(19) United States
(12) Patent Application Publication
(10) Pub. No.: US 2007/0191877 A1
(43) Pub. Date: Aug. 16, 2007

(54) Shape memory thin film embolic protection device

(76) Inventors: Minh Q. Dinh, Union City, CA (US); Scott M. Russell, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/227,052

(22) Filed: Sep. 15, 2005

Related U.S. Application Data
(60) Provisional application No. 60/610,898, filed on Sep. 17, 2004.

(51) Int. Cl. A61M 29/00

(52) U.S. Cl. 606/200

(57) ABSTRACT

A removable vascular frameless filter system for capture and retrieval of emboli while allowing continuous perfusion of blood. This system is useful for any percutaneous angioplasty, stenting, thrombolysis or tissue ablation procedure. The system may minimize the incidence of stroke, myocardial infarction or other clinical complications that may be associated with these procedures.

### CROSS REFERENCE TO RELATED APPLICATION

This application claims the benefit of U.S. Provisional Patent Application Ser. No. 60/610,898 filed Sep. 17, 2004.

### BACKGROUND OF THE INVENTION

I. Field of the Invention

The present invention relates to the treatment of vascular disease by either percutaneous angioplasty and stenting or surgery. More particularly, the present invention relates to a system that reduces macro- and micro-embolization during the treatment of vascular disease. Even more particularly, the present invention is directed to a collapsible filter device wherein the filter element comprises a shape memory thin film.

II. Discussion of the Related Art

A variety of surgical and non-surgical angioplasty procedures have been developed for removing obstructions from blood vessels. Balloon angioplasty utilizes a balloon-tipped catheter which may be inserted within a stenosed region of the blood vessel. By inflation of the balloon, the stenosed region is dilated. Stenting involves the permanent implantation of a metallic scaffold in the area of the obstruction, following balloon dilatation. The stent is often delivered on an angioplasty balloon, and is deployed when the balloon is inflated. Another alternative is the local delivery of medication via an infusion catheter. Other techniques, such as atherectomy, have also been proposed. In atherectomy, a rotating blade is used to shave plaque from an arterial wall. Finally, other techniques such as tissue ablation are sometimes performed to address electrical anomalies in heart rhythm. Surgery involves either removing the plaque from the artery or attaching a graft to the artery so as to bypass the obstructing plaque.

One problem common to all of these techniques is the accidental release of portions of the plaque or thrombus, resulting in emboli which can lodge elsewhere in the vascular system. Such emboli may be dangerous to the patient, and may cause severe impairment of the distal circulatory bed.

Depending upon the vessel being treated, this may result in a stroke, or myocardial infarction or limb ischemia.

Vascular filters or embolism traps for implantation into the vena cava of a patient are well known, being illustrated by, for example, U.S. Pat. Nos. 4,727,873 and 4,688,533. Additionally, there is a substantial amount of medical literature describing various designs of vascular filters and reporting the results of the clinical and experimented use thereof. See, for example, the article by Eichelter & Schenk entitled “Prophylaxis of Pulmonary Embolism,” Archives of Surgery, Vol. 97, Aug. 1968, pp. 348 et seq. See, also, the article by Greenfield, et al., entitled “A New lntracaval Filter Permitting Continued Flow and Resolution of Emboli”, Surgery, Vol. 73, No. 4, pp. 599-606 (1973).

Vascular filters are used, often during a postoperative period, when there is a perceived risk of a patient encountering a pulmonary embolus resulting from clots generated at the surgical site. Typically, the filter is mounted. in the vena cava to catch large emboli passing from the surgical site to the lungs.

The vascular filters of the prior art are usually permanently implanted in the venous system of the patient, so that even after the need for the filter has abated, the filter remains in place for the lifetime of the patient, absent surgical removal. U.S. Pat. No. 3,952,747 describes a stainless steel filtering device which is permanently implanted transvenously within the inferior vena cava. The filtering device is intended to treat recurrent pulmonary embolism. U.S. Pat. No. 4,873,978 describes a catheter device comprising a catheter body having a strainer mounted at its distal end. The strainer is shiftable between an opened configuration where it extends substantially across the blood vessel to entrap passing emboli, and a closed configuration where it retains the captured emboli during removal of the catheter. A mechanism actuable at the proximate end of the catheter body allows selective opening and closing of the strainer. Typically, the strainer is a collapsible cone having an apex attached to a wire running from the distal end to the proximate end of the catheter body.

Permanent implantation may be deemed medially undesirable, but it has been done because vascular filters are implanted in patients primarily in response to potentially life threatening situations. Accordingly, the potential disadvantages of permanent implantations of a vascular filter are often accepted.

Notwithstanding the usefulness of the above-described methods, a need still exists for an apparatus and method for preventing embolization associated with conventional surgery and interventional procedures. In particular, it would be desirable to provide a device which could be located within the vascular system to collect and retrieve portions of plaque and thrombus which have dislodged during the surgery or angioplasty procedure.

### SUMMARY OF THE INVENTION

The shape memory thin film embolic protection device of the present invention overcomes the disadvantages associated with currently utilized devices.

In accordance with one aspect, the present invention is directed to a removable percutaneously delivered filter system. The percutaneously delivered filter system comprises a delivery system, including a sheath and a filter section operatively associated with the delivery system. The filter section having a proximal end and a distal end. The proximal end having at least one opening allowing fluid to flow therethrough and the distal end having a multiplicity of pores for allowing fluid to flow therethrough and capturing particles of a predetermined size. The filter section being formed from a shape memory thin film material.

The present invention provides a vascular filter system useful in the surgical or interventional treatment of vascular disease. Macro-and micro-embolization may occur during percutaneous procedures such as angioplasty, which increases the risk of a minor or major stroke. The system of the present invention for reducing macro-and micro-embolization is very useful in helping to prevent the risk of stroke. However, this system would also be useful in any percutaneous angioplasty, stenting, thrombolysis or tissue ablation procedure, or surgical procedure where embolization is a risk. The vascular filter system of the present invention may decrease embolism while allowing brain, or other distal tissue, perfusion. The filters may be delivered to the location through a guide catheter which may be used for the entire procedure from crossing a lesion to deploying a stent.

The shape memory thin film embolic protection system offers a number of advantages, including easy delivery, compliance to various shaped vessels, low profile and increased radiopacity.

### BRIEF DESCRIPTION OF THE DRAWINGS

The present invention will be apparent upon consideration of the following detailed description, taken in conjunction with the accompanying drawings, in which the reference characters refer to like parts throughout, and in which:

FIG. 1 is a diagrammatic representation of a shape memory thin film embolic protection system inside of a vessel in accordance with the present invention.

FIG. 2 is a diagrammatic representation of a shape memory thin film embolic protection system in accordance with the present invention.

### DETAILED DESCRIPTION OF THE PREFERRED EMBODIMENTS

The present invention relates to a vascular filter system for use in percutaneous angioplasty and stenting as well as other vascular and non-vascular procedures as detailed herein, and provides for the prevention of distal embolism during vascular procedures. Further, the filter system of the present invention allows for distal perfusion while preventing embolization.

In accordance with one exemplary embodiment, the present invention is directed to a minimally invasive collapsible frameless filter for use in the field of medical procedures on vessels of the circulatory system. However, other uses are possible as the artisan will readily appreciate. Essentially, the frameless filter may be used in any organ in which there is a risk of debris becoming dislodged during a medical procedure. The frameless filter is preferably made of shape memory thin film, via physical vapor deposition or any other suitable process, that shapes like an expanded balloon of a balloon catheter. The device comprises multiple inlet openings at its proximal location to allow blood flow in and outlet openings, for example, a series of pores, at a distal location to filter blood clots and embolic material while allowing blood to exit from the filter. The filter, made from thin film, is a collapsible basket-shaped filter and can be made to contain no internal support structure. However, collapsible support frames may be utilized with the filter in alternate exemplary embodiments if desired. When the constraining sheath of the catheter is withdrawn during deployment, the shape memory properties of the filter allow it to reform to its programmed shape for capturing of embolic material within a vessel, for example. The flow of the blood assists in the deployment of the device and may enable more complete wall opposition. Essentially, the force of the blood flow facilitates the opening of the filter basket. Further, various filter basket geometries may be employed to optimize this deployment process. In other words, any number of suitable configurations may be utilized to comprise the filter basket and deployment thereof in order to accommodate the variety of blood vessels or other tubular organs posed by mammalian anatomy within which the filter may be used.

The filter device may be introduced into a vascular system in a collapsible configuration and delivered to its location through a guide catheter. When deployed the filter expands across a blood vessel such that blood passing through the blood vessel is delivered through the filter. A proximal inlet portion of the filter has multiple inlet openings to allow blood and embolic material to enter the filter, and a distal outlet portion of the filter has a plurality of small outlet openings (pores) to allow through-passage of blood, while retaining embolic material within the filter.

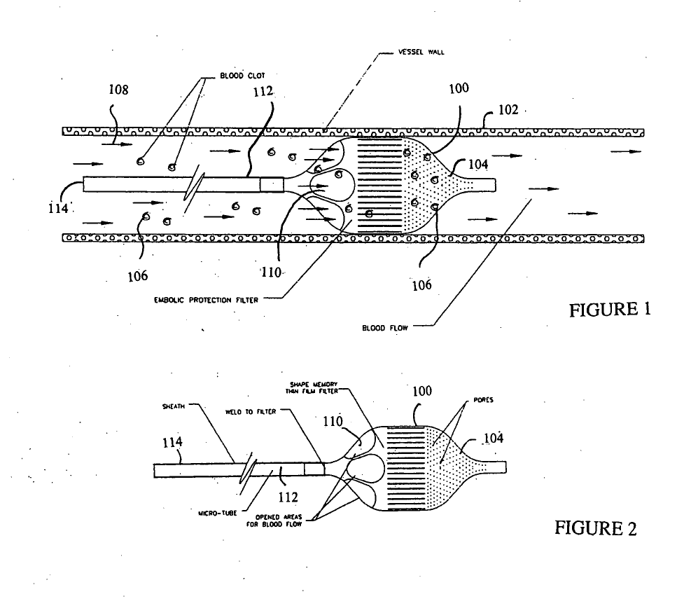

FIG. 1 illustrates an exemplary shape memory thin film embolic protection system 100 positioned within a vessel 102. The shape memory thin film embolic protection system 100 comprises a series of pores 104 at its distal end to capture embolic material or blood clots 106 flowing in the blood in the direction of arrows 108. The pores 104 of the thin film capture embolic material but allow blood to pass easily therethrough. The shape memory thin film embolic protection system 100 also comprises inlet openings 110 at its proximal end to allow blood to flow into the filter. The size and shape of the inlet openings 110 may comprise any suitable configuration depending on the application. The shape memory thin film embolic protection system 100 may be connected to the delivery system via any number of means. In the illustrated exemplary embodiment, the thin film section is fastened to a microtube 112 that is operatively associated with a catheter sheath 114. The fastening may be accomplished by any suitable means, including welding. FIG. 2 illustrates the same device, but not deployed in the vessel.

The thin film material, as stated above, may be fabricated from any number of suitable biocompatible materials, including metals, metal alloys, such as Nitinol, textiles, polymers, and composites. The material and design are subject to modification to ensure safety and efficacy. The material is preferably designed from a shape memory material. The material may comprise a super elastic or Martensitic shape memory material and, in the preferred embodiment, the material comprises nickel titanium alloy with about 50 to 60 weight percent nickel. The pores of the fabric are designed to capture particulate matter in the size ranging from about 50 μmto about 200 μm.

The shape memory thin film embolic protection system offers a number of advantages. The device is shaped like a non-compliant balloon that will preferably enhance one hundred percent wall opposition. The shape memory thin film with slotted pattern and no internal framework allows an extremely low profile configuration for delivery. The shape memory thin film with slotted pattern and no internal framework allows increased flexibility in the delivery sheath. The outlet openings may be designed to smaller size to allow a smaller capture profile. An increase of the longitudinal length of the device allows increased basket volume. An increase in radiopacity may be achieved by having larger surface areas.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

1. A removable percutaneously delivered filter system comprising:
a delivery system, including a sheath; and
a frameless filter section operatively associated with the delivery system having a proximal end and a distal end, the proximal end having at least one opening allowing fluid to flow therethrough and the distal end having a multiplicity of pores for allowing fluid to flow therethrough and capturing particles of a predetermined size, the filter section being formed from a shape memory thin film material.

2. A vascular filter system comprising:
a catheter delivery system, including a sheath; and
a collapsible frameless filter comprised of a biocompatible material that expands upon deployment as the sheath is retracted to oppose interior walls of a vessel, the filter having a proximal end with a plurality of openings therein, and a distal end with a plurality of openings therein, wherein blood flow assists the deployment of the filter.

3. The vascular filter system of claim 2, wherein the plurality of openings at the proximal end permit entry of blood into the filter, the plurality of openings at the distal end permit entry of the blood from the filter while embolic or other particulate materials are captured within the filter.

4. The vascular filter system of claim 3, wherein the size of the plurality of openings at the distal end of the filter determine a capture profile of the filter.

5. The vascular filter system of claim 4, wherein the size of the plurality of openings at the distal end of the filter capture embolic or particulate materials ranging from 50 μm to 200 μm.

6. The vascular filter system of claim 4, wherein the filter is radiopaque.

7. The vascular filter system of claim 6, wherein increasing a surface area of the filter increases radiopacity thereof.

8. The vascular filter system of claim 6, wherein the thin film comprising the filter is biocompatible Nitinol.

9. The vascular filter system of claim 6, wherein the thin film comprising the filter is comprised of any of the group of biocompatible materials consisting of metals, metal alloys, textiles, polymers and composites.

10. The vascular system of claim 8, wherein the filter is further comprised of a shape memory material.

11. The vascular filter system of claim 10, wherein the filter is further comprised of a super elastic or Martensitic shape memory material.

12. The vascular filter system of claim 11 wherein the filter is comprised of nickel titanium alloy with about 50 to 60 weight percent nickel.

13. The vascular filter system of claim 3, wherein a volume of the filter is increased by increasing a longitudinal length thereof.
